(19) United States
(12) Patent Application Publication
(10) Pub. No.: US 2008/0112846 A1
(43) Pub. Date: May 15, 2008

(54) Device for Sterilizing with Gaseous Plasma Formed from a Mixture of Nitrogen and Hydrogen

(75) Inventors: Francis Dieras, Bordeaux (FR); Andre Ricard, Toulouse (FR); Michel Sixou, Balma (FR); Sandrine Villeger, Toulouse (FR)

Correspondence Address: YOUNG & THOMPSON, 209 Madison Street, Suite 500, ALEXANDRIA, VA, 22314, US

(73) Assignee: SOCIETE POUR LA CONCEPTION DES APPLICATIONS DES TECHNIQUES ELECTRONIQUES- SATELEC, Merignac (FR)

(21) Appl. No.: 11/794,354

(22) PCT Filed: Dec. 27, 2005
(86) PCT No.: PCT/FR05/03287

§ 371 Date: Dec. 26, 2007

(30) Foreign Application Priority Data
Dec. 28, 2004 (FR) 0413984

(51) Int. Cl. A61L 2/20; B01J 19/08

(52) U.S. Cl. 422/33; 422/186.05

(57) ABSTRACT

A method for sterilizing objects (19), in particular medical or surgical instruments, includes subjecting a gas stream circulating in a chamber wherein an industrial vacuum is created to the action of an electric field so as to create a gaseous plasma whereof the post-discharge stream which is derived therefrom is admitted into a sterilizing chamber where it is contacted with the surface of the objects to be treated. The device is characterized in that the gas stream consists of a mixture of nitrogen, and of an amount of hydrogen less than 5%. It includes elements for heating the objects capable of bringing the latter, during treatment, to a temperature not less than 60° C.

The present invention relates to a sterilizing and cleaning device, intended in particular for medical or surgical instruments or apparatus. It also concerns a method for implementing said device.

It is known that sterilization consists of destroying, in a proportion laid down by the Pharmacopoeia, a significant number of pathogenic micro-organisms, viruses or proteins present on the inner or outer surface of items to be treated. Numerous methods exist which may achieve this result in more or less satisfactory manner.

In medical circles, sterilization is usually obtained by means of an autoclave in which the instruments to be sterilized are brought to a determined high temperature, in the order of 120° C., for determined periods of time with cycles laid down by legislation. It will be noted firstly that autoclaves are limited to the sterilization of items of small volume, which excludes their use for the sterilizing of conduits of equipment such as dialysers or dental treatment units. Also the application of a temperature higher than 100° C. to modern surgical instruments and accessories causes numerous constraints, and in particular prevents the sterilization of fragile objects or accessories containing parts in synthetic polymer materials for example on account of their heat-sensitivity.

This is why, in recent years, attention has been turned to methods with which it is possible to achieve sterilization at low temperature.

Sterilization devices have therefore proposed which have recourse to gases such as ethylene oxide, formaldehyde or hydrogen peroxide. These devices have proved to be of minor interest from a practical viewpoint insofar as they require a long desorption period incompatible with the need for rapid access to instruments, accessories or apparatus. Also, the gases used are not toxic-free and their efficacy is limited to some bacterial strains.

It has also been proposed to have recourse to gas plasma. It is recalled that for these techniques a gas is used which itself does not have bactericidal properties but is subjected to an electric field whose intensity is sufficiently high to cause its ionisation and the separation of its molecules, so that a plasma is obtained consisting of ions and electrons. It has been found that plasma has high bactericidal properties which have been used to ensure the sterilization of surgical and medical instruments. For this purpose, the plasma produced in this way enters a treatment chamber where it is contacted with the instruments it is desired to sterilize.

It is known however that if plasma has high sterilizing properties, it has the disadvantage of also having a destructive effect on some materials, such as synthetic plastic materials, which excludes its use for sterilizing numerous surgical or medical instruments.

It is also known that the gas produced downstream of the plasma, hereinafter called “post-discharge” gas, has sterilizing properties. This gas which is generated at the end of the plasma is no longer subjected to the effect of the electric field, which means that the electrons and ions forming the plasma disappear by recombination in the gas and after diffusion on the tube walls.

WO 00/72889 proposed a sterilization method having recourse in particular to a mixture of oxygen and nitrogen as plasma constituent gas. According to this technique it was found that the presence of atomic oxygen in the post-discharge gas has the effect of subjecting the polymers used in the surgical sector to an oxidation action, whether these are instrument parts such as dentistry hand-pieces, ultrasound equipment, endoscopes, catheters, seals, motors or various other apparatus.

In addition, during the formation of the gas plasma, the interaction of atomic oxygen with atomic nitrogen produces ultraviolet radiation whose bactericidal action is added to the effect of the post-discharge gas itself. This sterilization function produced by, ultraviolet radiation, while it is of interest in that it improves the sterilizing power of the device, has a serious drawback however in that the effects of the ultraviolet rays further add to the harsh nature of the treatment on the fragile parts of instruments.

Therefore, to avoid these disadvantages, the applicant proposed in patent FR 03.07799 a sterilizing device having recourse to a post-discharge gas derived from a gas plasma consisting exclusively of nitrogen. It was found that, during production of the plasma, the formation of ultraviolet rays was avoided thereby avoiding damage to the intactness of synthetic materials used in these instruments. It will be noted however that the device and method subject of this patent do not make it possible to neutralize the oxidizing nature of the oxidizing impurities existing in the sterilization chamber.

It will be noted that sterilizing devices intended for use in the medical sector, in particular in dentistry, firstly must not be too sophisticated from a practical viewpoint and secondly must be particularly accessible as regards cost i.e. in the order of the cost of currently used autoclaves for instrument sterilization. Said specifications therefore exclude having recourse to equipment of the type used by laboratories and require the use of accessories of so-called industrial type. Such accessories include the vacuum pump.

Vacuum pumps of so-called “industrial” type however have the disadvantage of not providing a sufficiently high vacuum (the residual pressure obtained is in the order of 10 Pa to 100 Pa) needed to avoid the presence of oxidizing impurities, including water vapour which, on plasma generation, produces OH⁻ radicals and air residues producing nitrogen oxides which evidently enter into the sterilizing chamber imparting oxidizing properties to the post-discharge gas. These oxidizing impurities attach themselves to the instruments to be sterilized and thereby start up a corrosion process.

The purpose of the present invention is to improve on the prior art by allowing the use in the inventive device of a vacuum pump of industrial type whilst further reducing the risks of oxidation of the instruments and equipment subjected to sterilization.

The subject-matter of the present invention is therefore a device for sterilizing items, surgical or medical instruments in particular, of the type in which a gas flow circulating in an enclosure in which a vacuum of industrial standard is set up, is subjected to the action of an electric field so as to create a gas plasma whose resulting post-discharge flow is caused to enter a sterilization chamber where it is contacted with the surface of the objects to be treated, characterized in that:
the gas flow consists of a mixture of nitrogen and a quantity of hydrogen of less than 5%,
it comprises means for heating said objects, able to bring the temperature thereof during treatment to at least 60° C.

The walls of the sterilization chamber may be made of a material having a low capacity for recombining the nitrogen and hydrogen atoms, such as glass and/or ceramic and/or a polymer. The objects to be sterilized may be arranged on a metal tray of a type which heats under the effect of the recombining of the nitrogen and hydrogen atoms, thereby ensuring the heating of the objects placed upon it. This tray which may be of brass in particular, may also be provided with its own heating means.

The electric field is preferably produced by a microwave generator, but could also be produced by direct or pulsed current discharges or by radiofrequencies.

In one embodiment of the invention, the sterilization chamber could consist of an autoclave, and this autoclave may form the means for heating the instruments to be sterilized.

Also the own means for generating the plasma may be contained in the autoclave door.

In one variant of embodiment of the invention, the heating of the objects contained in the sterilization chamber may be ensured by at least part of its walls which, for this purpose, consist of a material able to be heated through recombination of the nitrogen and hydrogen atoms. Heating of the objects may also be ensured by providing the walls of the sterilization chamber with additional heating means, electric in particular.

The present invention of is of particular interest in that it enables the sterilization of conduits and inner cavities of equipment and even of large volume equipment such as dental treatment units for example, dialysis equipment etc. For this purpose a post-discharge flow is injected via an orifice of this equipment and passes through its conduits and inner cavities, a flow which can be withdrawn by aspiration for example from another orifice.

For some equipment of reduced size which can be placed in a treatment chamber, the post-discharge flow could be caused to enter both the treatment chamber and the equipment via one of its orifices, and it could be removed by aspirating both from the treatment chamber and from the apparatus via a second orifice.

A further subject of the present invention is a method for sterilizing objects, surgical or medical instruments in particular, in which a plasma is created through the action of an electric field on a gas flow circulating in an enclosure in which an vacuum of industrial standard is set up, and the resulting post-discharge flow is contacted with the surface of the objects to be treated, characterized in that:
as gas flow, a mixture of nitrogen and a quantity of hydrogen of less than 5% is used,
heating of the objects to be treated is ensured to a temperature of at least 60° C.

In accordance with the invention, it is possible to raise the temperature of the instruments during treatment, this temperature increase possibly being obtained by heating the tray carrying the objects or by heating the sterilization chamber, but also by recombining the atoms of the post-discharge gas on the surfaces of the tray and/or sterilization chamber.

It is known that the nitrogen and hydrogen atoms produced by the post-discharge react by atomic recombination on the surface of the objects to be treated, and that these reactions are exothermal. Therefore, it was determined that, under the tested experimental conditions, namely: pressure of 665 Pa, microwave generator power of 100 W and flow rate of 1 l/min, the surface temperature of the materials reached 80° C. for brass, 55° C. for steel, 60° C. for aluminium, 55° C. for titanium, 40° C. for ceramic and 37° C. for glass.

Yet it was found, regarding the bacterium Escherichia Coli, that a temperature of 60° C. is required to induce a 10⁵ decrease in the bacterial population after 40 minutes' exposure to nitrogen post-discharge. Therefore, to ensure efficient sterilization of instruments irrespective of type, their surface needs to be brought to a minimum temperature of 60° C. during sterilization.

An embodiment of the invention is described below as a non-limitative example with reference to the appended drawing in which:

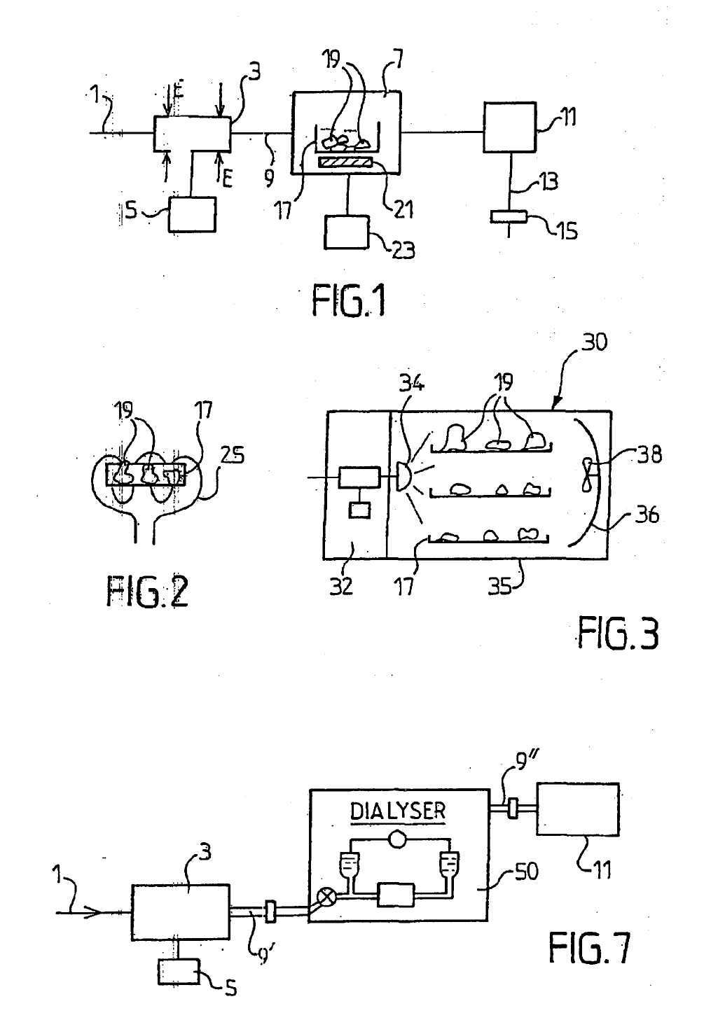

FIG. 1 is a schematic view of a sterilizing device of the invention,

FIG. 2 is a variant of embodiment of the sterilization device shown FIG. 1,

FIG. 3 is a schematic view of a variant of embodiment of the inventive device,

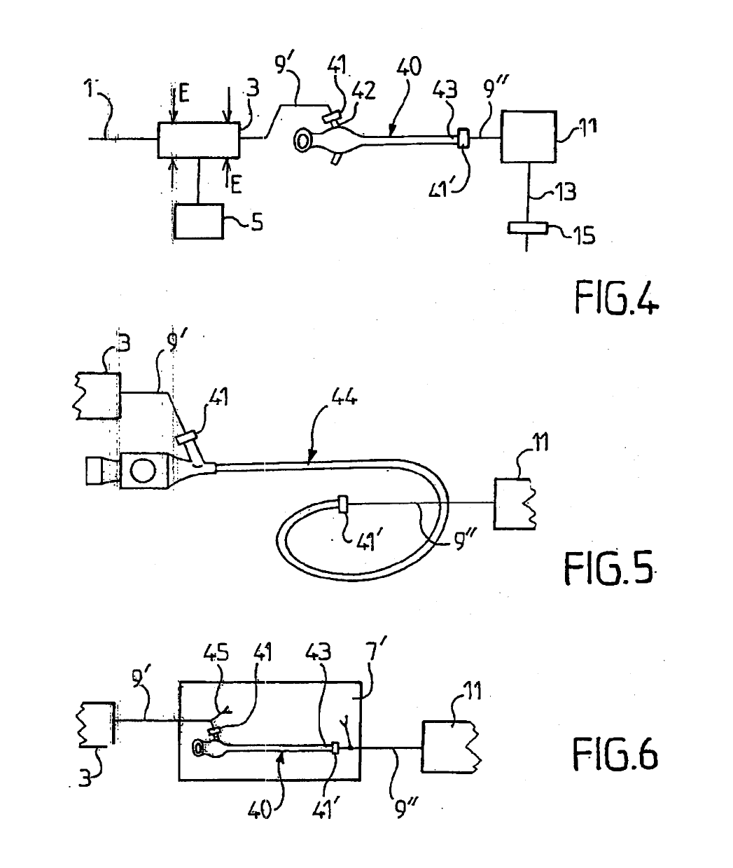

FIGS. 4 and 5 are schematic views of two applications of the inventive device to the sterilization of conduits and inner cavities of an endoscope and fibroscope,

FIG. 6 is a schematic view of an example of sterilization of the outer surface, conduits and inner cavities of an apparatus.

FIG. 7 is a schematic view of an application of the inventive device to the sterilization of the conduits and inner cavities of dialysis equipment.

FIG. 1 very schematically shows a gas plasma sterilization device of the invention. This device comprises an inlet pipe 1 for a gas flow consisting of a mixture of nitrogen and hydrogen which passes through a vacuum enclosure subjected to the action of en electric field generator formed by a microwave generator 3 of 2.45 GHz whose power is regulated by control means 5. The post-discharge gas generated by the plasma produced (in known manner) is brought into a treatment chamber 7 via a pipe 9. This treatment chamber 7 is arranged in the plasma post-discharge zone and communicates with a vacuum pump 11. This pump drives the post-discharge gas into the treatment chamber 7 and ensures the evacuation of the gases towards the outside via pipe 13 provided with appropriate filters 15.

The treatment chamber 7 comprises a metal tray 17 intended to carry the objects 19 to be sterilized.

The tray 17 is provided with heating means 21 whose temperature is controlled by a control device 23. These heating means may in particular consist of an electric resistance or, as shown FIG. 2, of induction heating means 25.

As shown FIG. 3, the treatment chamber may consist of an autoclave of the type used to sterilize surgical or medical instruments.

In this figure, the autoclave 30 is formed of an enclosure 35, of substantially parallelepiped shape, which is closed on one of its sides by a pivoting door 32. This pivoting door is of sufficient thickness to contain the various elements required for generating the plasma. On its front side it comprises an outlet nozzle 34 for the post-discharge gas intended to supply the inside of the enclosure. This nozzle 34 may advantageously end in one or more injectors making it possible in particular to homogenize the flow of the post-discharge gas.

In the embodiment shown FIG. 3 the enclosure 35, on its wall opposite the door 32, is provided with a “reflector” 36 and a ventilator 38 which contribute towards homogenizing the post-discharge gas within the enclosure 35. Said arrangement is of interest in that it provides the user with a multi-function autoclave, namely a conventional autoclave function and a function in which sterilization is made by a post-discharge gas at low temperature. Therefore, in relation to the objects to be sterilized, the user has the option of using the most appropriate sterilization mode.

In this variant of embodiment of the invention, the autoclave may be used to bring the temperature of the objects to be sterilized to the desired temperature.

It was found that it is possible to obtain a post-discharge gas having bactericidal properties from a gas flow supply consisting of a mixture of nitrogen and hydrogen without having recourse to atomic oxygen as is taught in the prior art.

It was determined that a post-discharge gas obtained from a gas flow consisting of a mixture of nitrogen and hydrogen has a marked biocide effect on bacteria.

It was also found that the extent of the biocide effect obtained is related to the nature of the carrier tray used and to the temperature to which it is brought during the sterilization operation.

In addition, it was found that the effect of the mixture of nitrogen and hydrogen is to produce NHx radicals (where x=1,2,3) which proved to be active for deoxidisation and sterilization.

It will therefore be understood that with the present invention it is possible to increase the efficacy of plasma sterilization devices by having recourse to nitrogen as plasma generating gas.

The hydrogen atoms produced in this way ensure a twofold function, i.e. firstly they generate a reducing reaction of the oxidizing gas impurities associated with the instruments to be sterilized and whose decomposition by the plasma could corrode these instruments, and secondly these atoms and the NHx radicals which become added to the nitrogen atoms produce a surface chemistry on the instruments to be sterilized. It was found that this surface chemistry disorganizes the organic macromolecules and, together with a slight temperature increase in the order of 60° C., destroys the microorganisms by decomposing them. The resulting desorption gases are evacuated outside the enclosure by pumping.

With the present invention, it is also possible to ensure the sterilization of equipment parts which, on account of their type or size, cannot be sterilized in sterilizers of conventional type.

Therefore, as shown FIG. 5, the sterilization device shown FIG. 1 was applied to the sterilization of an endoscope 40. For this purpose one of its inlet orifices 42 is connected by a connector 41 to a pipe 9′ connected to the output of the plasma generator 3 so that the post-discharge gas forms inside a sterilization chamber formed by the conduits and inner cavities of the endoscope 40. Its outlet end 43 is connected via a connector 41′ to a pipe 9″ connected to a vacuum pump 11. According to the invention, the post-discharge gas which passes through the inside of the endoscope cavities will ensure the sterilization thereof.

It will be noted that said mode of use is of particular interest, firstly regarding its ease of use by the practitioner and secondly in that it can ensure the sterilization of apparatus which may contain parts on its outer surface that are made in materials not resisting the temperatures required by sterilization of conventional type.

As shown FIG. 6 therefore, an identical sterilization device may be applied to other types of instruments, in particular to a fibroscope 44.

Evidently it is possible according to the invention to sterilize the entirety of the instrument, namely its conduits and cavities as well as its outer surface, if so desired, by arranging it inside a sterilization chamber 7′ which is in communication via a pipe 9′ with the plasma generator 3, this pipe being connected to an inlet of the endoscope via a connector 41 and also being in communication via a nozzle 45 with the inside of the sterilization chamber 7′ in which the post-discharge gas is formed, the outlet 43 of the endoscope 40 and the inner volume of the sterilization chamber being connected to a vacuum pump 11 as shown FIG. 7.

It is also possible to use the inventive device to ensure the sterilization of the conduits and inner volumes of a dental treatment unit by connecting an intake of this unit to the supply of the post-discharge gas and its outlet to a vacuum pump.

A further application of the invention of particular interest consists of sterilizing dialysis equipment as shown FIG. 8. The dialysis unit 50 is connected via its intake to a supply pipe 9′ for the post-discharge gas and its outlet is connected to a vacuum pump 11.

1-14. (canceled)

15. Device for sterilizing objects (19, 40, 42, 50), in particular medical or surgical instruments, comprising an enclosure having an inlet pipe (1) for receiving a gas and an outlet pipe for evacuating the gas, a vacuum pump (11) for setting up a vacuum of industrial standard in said enclosure, a microwave generator (3) to subject a received gas flow to the action of an electric field so as to create a plasma gas, a sterilization chamber (7) for receiving a post-discharge flow resulting from said plasma gas and intended for receiving said objects, characterized in that it comprises:
a gas flow source which consists of a mixture of nitrogen and a quantity of hydrogen of less than 5%,
heating means (21) for said objects, able to bring the temperature thereof during treatment to at least 60° C.

16. Device as in claim 15, characterized in that the walls of the sterilization chamber (7) consist of a material having a low capacity for recombining the nitrogen and hydrogen atoms.

17. Device as in claim 16, characterized in that the wall of the sterilization chamber consists of glass and/or ceramic and/or a polymer.

18. Device as in claim 16, characterized in that the sterilization chamber consists of an autoclave (30).

19. Device as in claim 18, characterized in that the means for heating said objects consist of the autoclave's own heating means.

20. Device as in claim 15, characterized in that it comprises in a metal tray (17) for receiving the objects to be handled, the nature of the metal tray being chosen such that, under the effect of the recombination of the nitrogen and hydrogen atoms, said tray heats and ensures the heating of the objects (19) it contains.

21. Device as in claim 20, characterized in that the tray is made of brass.

22. Device as in claim 20, characterized in that the tray is provided with heating means (21).

23. Device as in claim 19, characterized in that the walls of the sterilization chamber (7) comprise at least in part a material able to heat through recombination of the nitrogen and hydrogen atoms.

24. Device as in claim 16, characterized in that the walls of the sterilization chamber (7) are provided with additional heating means, electric in particular.

25. Device for sterilizing according to claim 15, for an object of the apparatus type (40, 42, 50) comprising conduits or inner cavities which it is desired to sterilize and which are in communication with the outside via inlet and outlet orifices, characterized in that it comprises means for injecting post-discharge flow via an orifice of such apparatus through its conduits and inner cavities, this flow being expelled via the other orifice.

26. Device as in claim 25, characterized in that the apparatus to be sterilized (40) is arranged in a treatment chamber (7′) through which the post-discharge gas also passes.

27. Method for sterilizing objects, medical or surgical instruments in particular, wherein a plasma is created through the action of an electric field on a gas flow circulating in an enclosure in which a vacuum of industrial standard is set up, and the resulting post-discharge flow is contacted with the surface of the objects to be treated, characterized in that:
as gas flow, a mixture of nitrogen and a quantity of hydrogen less than 5% is used,
the objects to be treated are heated to a temperature of at least 60° C.
